(19) United States
(12) Patent Application Publication
(10) Pub. No.: US 2024/0165302 A1
(43) Pub. Date: May 23, 2024

(54) RECAPITULATING THE HEMATOPOIETIC NICHE TO RECONSTITUTE IMMUNITY

(71) Applicants: President and Fellows of Harvard College, Cambridge, MA (US); The General Hospital Corporation, Boston, MA (US)

(72) Inventors: Nisarg J. Shah, San Diego, CA (US); Ting-Yu Shih, Brookline, MA (US); Angelo S. Mao, Cambridge, MA (US); David J. Mooney, Cambridge, MA (US); David T. Scadden, Weston, MA (US)

(73) Assignees: President and Fellows of Harvard College, Cambridge, MA (US); The General Hospital Corporation, Boston, MA (US)

(21) Appl. No.: 18/224,444

(22) Filed: Jul. 20, 2023

Related U.S. Application Data
(63) Continuation of application No. 16/075,937, filed on Aug. 6, 2018, now Pat. No. 11,752,238.
(60) Provisional application No. 62/292,288, filed on Feb. 6, 2016.

(51) Int. Cl. A61L 27/54; A61L 27/20; A61L 27/52; A61L 27/56; C12N 5/0789

(52) U.S. Cl. CPC A61L 27/54; A61L 27/20; A61L 27/52; A61L 27/56; C12N 5/0647; A61L 2300/414; A61L 2300/426; A61L 2300/45; A61L 2400/18; A61L 2430/02; C12N 2501/155; C12N 2533/74

(57) ABSTRACT

Disclosed are compositions and related methods of recapitulating bone marrow stroma using scaffold materials (e.g., a porous alginate hydrogel scaffold) containing one or more cellular differentiation factors, and one or more growth factors. Such methods and compositions promote the formation of an ectopic nodule or site that can improve transplanted cell engraftment and selectively drive the development of lymphocytes and the reconstitution of the adaptive immunity after hematopoietic stem cell transplant.

### RELATED APPLICATION

This application claims the benefit of U.S. Provisional Application Ser. No. 62/292,288, filed Feb. 6, 2016, the entire teachings of which are incorporated herein by reference.

### GOVERNMENT SUPPORT

This invention was made with government support under Grant Nos. NIH HL129903, NIH EB015498, and NIH EB014703 awarded by the National Institutes of Health and NSF 1000099416 awarded by the National Science Foundation. The government has certain rights in the invention.

### BACKGROUND OF THE INVENTION

Long-term immunodeficiency in patients receiving a hematopoietic stem cell transplant (HSCT) remains one of the most serious impediments in managing life-threatening diseases of the blood or bone marrow, such as multiple myeloma and leukemia. Before transplantation, the recipient undergoes a conditioning cytotoxic radiation and chemotherapy regimen to destroy the diseased cells. A side effect of the conditioning process is severe lymphopenia as a result of T- and B-cell destruction of the adaptive immune system. The profound post-transplant immunodeficiency, which is characterized by a dramatic reduction in the number of T- and B-cells and a reduction in their diversity, can persist for one to two years. Immunodeficiency related severe opportunistic infections (˜30%), cancer relapse (>50% for acute myeloid leukemia) and graft-versus-host disease (GVHD) (˜40%) are the most common complications and cause of morbidity and mortality in patients receiving a HSCT.

Needed are novel compositions and methods that are useful for improving the reconstitution of the immune system post-HSCT. Also needed are compositions and methods that are able to reduce the risk associated with HSCT and improve patient outcomes.

### SUMMARY OF THE INVENTION

Disclosed herein are novel compositions and related methods that are useful for aiding in the reconstitution of the immune system of a subject, for example, post-stem cell transplant. Such compositions may be administered to a subject to increase the engraftment of transplanted stem cells and progenitor cells and thereby aid in the reconstitution of the subject's immune system.

In certain embodiments, the compositions disclosed herein comprise one or more scaffold materials (e.g., a porous implantable scaffold material), which may be administered to or otherwise implanted in a subject (e.g., implanted subcutaneously at one or more site in or around the lymph nodes of a subject). Such compositions may further comprise one or more growth factors, one or more homing factors and one or more differentiation factors.

In certain aspects, the scaffold material is or comprises a hydrogel (e.g., cryogel). In certain embodiments, the scaffold material is ostcoinductive. In certain embodiments, the scaffold material comprises alginate (e.g., anionic alginate). In some embodiments, the scaffold material is selected from the group consisting of polylactic acid, polyglycolic acid, PLGA polymers, alginates and alginate derivatives, polycaprolactone, calcium phosphate-based materials, gelatin, collagen, fibrin, hyaluronic acid, laminin rich gels, agarose, natural and synthetic polysaccharides, polyamino acids, polypeptides, polyesters, polyanhydrides, polyphosphazines, poly(vinyl alcohols), poly(alkylene oxides), poly(allylamines)(PAM), poly(acrylates), modified styrene polymers, pluronic polyols, polyoxamers, poly(uronic acids), poly(vinylpyrrolidone) and any combinations or copolymers thereof.

The compositions and scaffold materials disclosed herein are useful as a carrier or vehicle to deliver one or more growth factors (e.g., to deliver one or more growth factors in vivo). Following administration or implantation of the compositions, the one or more growth factors contained in such compositions promote the formation of tissue (e.g., bony tissue) on or around the administered scaffold material, thereby forming a nodule. In certain aspects, one or more of the growth factors comprise a bone morphogenetic protein (BMP) (e.g., BMP-2). For example, one or more of the growth factors may be selected from the group consisting of BMP-2, BMP-4, BMP-6, BMP-7, BMP-12, BMP-14, TGF-β, IGF-1, FGF-2, and PDGF. In some embodiments, one or more of the growth factors may be encapsulated in the scaffold material (e.g., encapsulated and released from the scaffold material over about 7-14 days). In some embodiments, such one or more of the growth factors (e.g., BMP-2) are released from the scaffold material over an extended period of time (e.g., about 7-30 days or longer, about 17-18 days).

The compositions and scaffold materials disclosed herein may also serve as a carrier or vehicle for one or more differentiation factors. In certain aspects, the contemplated differentiation factors induce or promote the differentiation of a stem cell or progenitor cell (e.g., a transplanted HSC) to one or more desired cell types. For example, one or more of the differentiation factors may be incorporated into the compositions disclosed herein to promote the differentiation of transplanted stem cells or progenitor cells to lymphoid lineage cells. In some embodiments, one or more of the differentiation factors bind to a Notch receptor (e.g., one or more of the differentiation factors may bind to a Notch receptor selected from the group consisting of Notch-1, Notch-2, Notch-3 and Notch-4). In certain aspects, one or more of the differentiation factors are selected from the group consisting of Delta-like1, Delta-like3, Delta-like4, Jagged1 and Jagged2.

In some embodiments one or more of the differentiation factors comprise a cytokine (e.g., a cytokine selected from the group consisting of interleukin-7 (IL-7) and interleukin-15 (IL-15)). In some embodiments one or more of the cytokines may be encapsulated in the scaffold material. In certain aspects, one or more of the cytokines are released from the scaffold material over an extended period of time (e.g., about 7-30 days or longer, about 17-18 days).

In certain embodiments, one or more of the differentiation factors are covalently bound to the scaffold material. For example, rather than being released from an alginate scaffold material, a differentiation factor may be covalently bound to the alginate backbone and retained within the nodule that forms following implantation of the composition in the subject. By covalently binding or coupling a differentiation factor to the scaffold material, such differentiation factor will be retained within the nodule that forms following administration of the composition to a subject, and thus will be available to promote the differentiation of a transplanted stem cells or progenitor cell, as contemplated herein. In certain embodiments, one or more differentiation factors are conjugated to the scaffold material utilizing N-hydroxysuccinimide (NHS) and 1-ethyl-3-(3-dimethylaminopropyl)carbodiimide hydrochloride (EDC) chemistry. Any methods of covalently binding or coupling differentiation factors known in the art may be used and are not limited. See “Bioconjugate Techniques Bioconjugate Techniques (Third Addition)”, Greg T. Hermanson, Academic, Greg T. Hermanson, Academic Press, 2013 Press, 2013.

In certain embodiments, the compositions and scaffold materials disclosed herein comprise one or more homing factors. In certain aspects, the inclusion of such homing factors in the compositions disclosed herein promotes the homing of transplanted stem cells and/or progenitor cells (e.g., HSCs) to the implanted composition or nodule. In certain aspects, such homing factors promote the infiltration of transplanted stem cells or progenitor cells (e.g., HSCs) to the implanted composition or nodule. In some embodiments, one or more of the homing factors comprise stem cell differentiation factor (SDF-1). In certain embodiments, one or more of the homing factors are encapsulated in the material. In certain embodiments, one or more of the homing factors are released from the material over an extended period of time (e.g., about 7-30 days or longer, about 17-18 days).

In some embodiments about 0.01 nmol to 1000 nmol, about 0.1 nmol to 100 nmol, or 1 nmol to about 10 nmol of one or more growth factors, one or more homing factors and one or more differentiation factors is conjugated to the scaffold material.

In some embodiments, the present inventions are directed to methods of aiding or supporting the reconstitution of the immune system of a subject in need thereof, such methods comprising a step of administering to the subject a composition comprising a scaffold material comprising one or more growth factors which promote formation of tissue on or around the administered scaffold material to form a nodule; one or more homing factors which promote the infiltration of transplanted stem cells or progenitor cells to the nodule; and one or more differentiation factors which promote the differentiation of the transplanted stem cells or progenitor cells to lymphoid lineage cells, thereby aiding or supporting the reconstitution of the immune system of the subject. In some embodiments, the subject has undergone cytoxic radiation and/or chemotherapy to treat a blood disorder.

In certain embodiments, the inventions disclosed herein are directed to methods of forming an ectopic hematopoietic stem cell niche in a subject in need thereof, such methods comprising a step of administering to the subject a composition comprising a scaffold material comprising one or more growth factors which promote formation of tissue on or around the administered scaffold material to form a nodule; one or more homing factors which promote the infiltration of transplanted hematopoietic stem cells to the nodule; and one or more differentiation factors which promote the differentiation of the transplanted stem cells or progenitor cells to one or more lymphoid or myeloid lineage cells, thereby forming an ectopic hematopoietic stem cell niche in the subject.

In yet other embodiments, the present inventions are directed to methods of improving engraftment of a transplanted hematopoietic stem cell in a stem cell niche of a subject in need thereof, the method comprising administering to the subject a composition comprising a scaffold material comprising one or more growth factors which promote formation of tissue on or around the administered scaffold material to form a nodule; one or more homing factors which promote the infiltration of the transplanted hematopoietic stem cells to the nodule; and one or more differentiation factors which promote the differentiation of the transplanted stem cells or progenitor cells to one or more lymphoid or myeloid lineage cells (e.g., one or more of CD4+, CD8+, and Mac-1+/GR-1+ lymphoid or myeloid lineage cells), thereby improving engraftment of the transplanted hematopoietic stem cell in the stem cell niche of the subject.

In certain embodiments, the inventions disclosed herein relate to methods of increasing the sites (e.g., increasing the number or volume of one or more sites) for transplanted stem cells and progenitor cells to engraft in a subject in need thereof, such methods comprising administering to the subject a composition comprising a scaffold material comprising one or more growth factors which promote formation of tissue on or around the administered scaffold material to form a nodule; one or more homing factors which promote the infiltration of the transplanted stem cells and progenitor cells to the nodule; and one or more differentiation factors which promote differentiation of the transplanted stem cells and progenitor cells to one or more lymphoid or myeloid lineage cells (e.g., one or more of CD4+, CD8+, and Mac-1+/GR-1+ lymphoid or myeloid lineage cells), thereby increasing sites for the transplanted stem cells and progenitor cells to engraft in the subject.

In certain aspects, contemplated scaffold materials for use in accordance with the methods of the present invention comprise a hydrogel material (e.g., cryogel material). In some embodiments, the scaffold material comprises alginate (e.g., a scaffold material comprising anionic alginate). In some embodiments, the scaffold material is osteoinductive. In certain embodiments, the scaffold material is selected from the group consisting of polylactic acid, polyglycolic acid, PLGA polymers, alginates and alginate derivatives, polycaprolactone, calcium phosphate-based materials, gelatin, collagen, fibrin, hyaluronic acid, laminin rich gels, agarose, natural and synthetic polysaccharides, polyamino acids, polypeptides, polyesters, polyanhydrides, polyphosphazines, poly(vinyl alcohols), poly(alkylene oxides), poly(allylamines)(PAM), poly(acrylates), modified styrene polymers, pluronic polyols, polyoxamers, poly(uronic acids), poly(vinylpyrrolidone) and any combinations or copolymers thereof.

The compositions and methods disclosed herein generally contemplate a step of administering the compositions to the subject. In certain aspects, such an administration step comprises implanting the composition in the subject (e.g., the composition may be implanted subcutaneously). For example, in some embodiments, the compositions disclosed herein may be subcutaneously administered to a subject at one or more sites in or around a lymph node in advance of a stem cell transplant (e.g., about 5 days, 7 days, 10 days, 14 days, 18 days, 21 days, 24 days, 28 days, 30 days, 35 days, 42 days or more prior to a stem cell transplant).

The compositions for use in accordance with any of the foregoing methods may comprise one or more growth factors. For example, such growth factors (e.g., BMP-2) may be encapsulated by the scaffold material and promote the formation of a nodule (e.g., a bony nodule) in or around the administered composition. In certain aspects, one or more of the growth factors comprise a bone morphogenetic protein (BMP). In some embodiments, one or more of the growth factors are selected from the group consisting of BMP-2, BMP-4, BMP-6, BMP-7, BMP-12 and BMP-14. In certain aspects, one or more of the growth factors are encapsulated in the material. In certain aspects, one or more of the growth factors are released from the material over an extended period of time (e.g., about 7-30 days or longer).

The compositions for use in accordance with any of the foregoing methods may also comprise one or more differentiation factors. Such differentiation factors may be used to promote the differentiation of transplanted stem cells and progenitor cells (e.g., transplanted HSCs) in vivo. For example, in some aspects, one or more of the differentiation factors promote lymphopoiesis of transplanted stem cells and progenitor cells (e.g., transplanted HSCs) in vivo.

In certain aspects, one or more of the differentiation factors comprise a composition that binds to a Notch receptor. In certain aspects, the Notch receptor is selected from the group consisting of Notch-1, Notch-2, Notch-3 and Notch-4. In some embodiments, one or more of the differentiation factors are selected from the group consisting of Delta-like1, Delta-like3, Delta-like4, Jagged1 and Jagged2.

In some embodiments, one or more of the differentiation factors comprise a cytokine. For example, one or more of the differentiation factors may be selected from the group of cytokines consisting of IL7 and IL-15.

In some embodiments, one or more of the differentiation factors (e.g., a cytokine) are encapsulated in the material. In some embodiments, one or more of the differentiation factors (e.g., a cytokine) are released from the material over about 7-30 days. Alternatively, in some embodiments, one or more of the differentiation factors are covalently bound to the material.

The compositions for use in accordance with any of the foregoing methods may also comprise one or more homing factors. In some embodiments, one or more of the homing factors comprise stem cell differentiation factor (SDF-1). In certain embodiments, one or more of the homing factors are encapsulated in the material. In some embodiments, one or more of the homing factors are released from the material over about 7-30 days.

The compositions and methods disclosed herein are suitable for administration or implantation in a subject (e.g., a subject in need thereof). For example, such compositions and methods may be useful for the treatment of a subject that has undergone a stem cell transplant and/or a subject that is immunocompromised.

The above discussed, and many other features and attendant advantages of the present inventions will become better understood by reference to the following detailed description of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 depicts a general overall approach of an embodiment of the present invention. As depicted in FIG. 1, a bone nodule-forming injectable protein-polymer hydrogel recruits transplanted hematopoietic stem cells (HSCs) in vivo. Recruited cells HSCs were presented with differentiation factors present in the hydrogel to drive their differentiation into lymphocytes.

FIGS. 2A-2E depict an ectopic bone nodule formed in accordance with the present inventions. FIG. 2A illustrates the release of a growth factor (BMP-2) encapsulated within the hydrogel, while and FIG. 2B (top) graphically illustrate the release of a growth factor (BMP-2) encapsulated within the hydrogel and which is released over about one to two weeks, FIG. 2B (middle) shows the release of both BMP-2 and the differentiation factor delta-like4 (DLL-4) from the hydrogel over about one to two weeks, and FIG. 2B (bottom) shows the cumulative amounts of BMP-2 and the differentiation factor interleukin-7 (IL-7) released from the hydrogel over about one to two weeks. FIG. 2C depict a subcutaneous bone nodule (red arrow). FIG. 2D depicts a three-dimensional micro-computed tomography image of the bone nodule. FIG. 2E depicts a Safranin-O stained histology section of an excised bone nodule demonstrating bone alginate and marrow.

FIGS. 3A and 3B depict hydrogel-mediated lymphopoiesis in accordance with the present invention. FIG. 3A demonstrates that isolated LKS cells cultured in vitro in the presence of bone nodule-forming hydrogel exhibited differentiation in the presence of immobilized, tethered DLL-4 (n=9, ***P<0.001,**P<0.01, ns=not significant). FIG. 3B illustrates that in vivo, the ectopic bone nodule accelerates reconstitution of T-cells (CD3+) and B-cells (B220+) in sub-lethally irradiated mice. No change in the reconstitution kinetics of the myeloid cell (Mac-1+Gr-1+) population.

FIGS. 4A and 4B depict the characterizing immune reconstitution after hematopoietic stem cell transplant (HSCT). As shown in FIG. 4A, the BMP-2/DLL-4 containing hydrogel significantly accelerated T-cell and B-cell recovery post-HSCT with a T-cell and B-cell depleted graft. FIG. 4B depicts the results of an analysis of the immune repertoire 2 weeks after HSCT and indicates that the greater VJ recombination events occurred in CD3+ T-cells with the BMP-2/DLL-4 hydrogel.

FIG. 5 shows conventional HBSC treatment for blood disorders including leukemia and myeloma. The upper rows show a patient with a blood disorder undergoing radiation treatment to eradicate host blood cells, followed by reconstitution of the blood and immune system with a blood stem cell transplant. The lower row shows a sample of blood from a blood disorder patient; normal and irradiated bone marrow; and the repopulation of myeloid cells within weeks of the blood stem cell transplant and repopulation of T- and B-cells months to years after the transplant.

FIG. 6 illustrates a schematic wherein HSC and a hydrogel with DLL-4 as described herein are implanted in a mouse to accelerate immune reconstitution.

FIG. 7 shows the components for a hydrogel as described herein. The top row shows DLL4-PEG₂ₖ-MA and BMP-2. The middle row illustrates methylation of Alginate and PEG using EDC/NHS chemistry and 2-aminoethyl methacrylate (AEMA). The bottom row illustrates formation of a macroporous cryogel as described herein via cryo-polymerization.

FIG. 8A shows a schematic of culturing and analyzing blood stem cells in vitro. FIG. 8B illustrates the degree of functionalization with COOH and relative cell abundance for T-cells, B-cells, Myeloid cells, CLP and CMP.

FIG. 9 shows that distinct CLP/T-cells dynamics in vitro are DLL-4 dependent. The left side graph provides data for cells contacted with a hydrogel with BMP-2 and DLL-4. The right side graph provides data for cells contacted with a hydrogel with BMP-2.

FIG. 10 shows a schematic of cell transplantation using GFP+ cells into an irradiated mouse.

FIG. 11 shows that BMP-2 release enhances transplanted cell numbers in cryogel.

FIG. 12 shows that BMP-2 and DLL-4 enhance CLP cell numbers in cryogel.

FIG. 13A shows BMP-2 induces an ectopic bone marrow nodule. FIG. 13B is a picture of an ectopic bone marrow nodule grown by methods disclosed herein. FIG. 13C is a photograph of a bone nodule in subcutaneous tissue grown by methods disclosed herein.

FIG. 14 shows numbers of T-cells, B-cells and Myeloid cells 0-60 days after irradiation in mice with transplant only (left graph), transplant plus hydrogel with BMP-2 (middle graph), and transplant plus hydrogel with BMP-2 and DLL-4 (right graph).

FIG. 15 illustrates that sequencing of CDR's 1-3 in TCRβ can provide a snapshot of cell diversity.

FIG. 16 shows TCR repertoire analysis for non-irradiated, transplant only, transplant plus hydrogel with BMP-2, and transplant plus hydrogel with BMP-2 and DLL-4.

FIG. 17A shows a schematic for sjTREC analysis to measure thymic output.

FIG. 17B shows sjTREC/mg thymus for non-irradiated, transplant only, transplant plus hydrogel with BMP-2, and transplant plus hydrogel with BMP-2 and DLL-4.

FIG. 18A shows a schematic for a sublethal viral injection challenge after immune reconstitution for 1 month. FIG. 18B shows survival post viral challenge for non-irradiated, transplant only, transplant plus hydrogel with BMP-2, and transplant plus hydrogel with BMP-2 and DLL-4 mice.

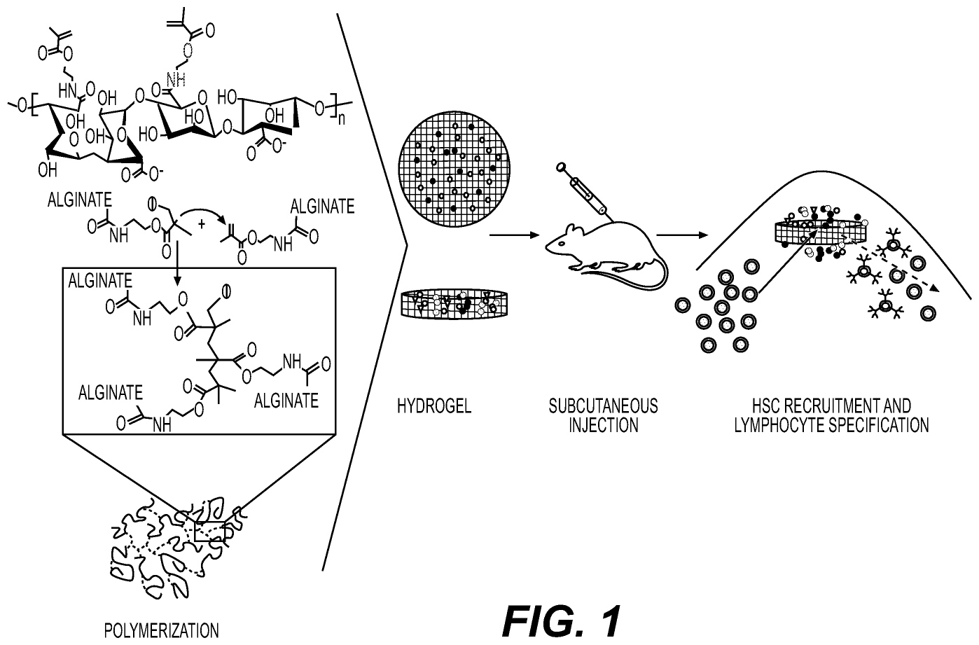

### DETAILED DESCRIPTION OF THE INVENTION

A fundamental challenge after hematopoietic stem cell transplant (HSCT) concerns the generation of new immunological responses, while avoiding an over-exuberant reaction that could result in autoimmune disorders. At the stem cell level, the immune system arises from hematopoietic stem cells (HSCs) which reside in bone marrow niches and comprise the cells and the surrounding matrix. In addition to its role as a primary lymphoid organ through the support of lymphoid development, the bone marrow acts as a host for various mature lymphoid cell types. The bone marrow influences the regenerative capacity and differentiation potential of HSCs into immune cells and provides the progenitor cell population for new T-cells and B-cells.

The hematopoietic stem cell transplant (HSCT) conditioning process damages the bone marrow niche and its ability to support the engrailment of donor cells which directly impacts the reconstitution of the immune system. Consequently, immunomodulatory small molecules, therapeutic proteins and infusions of purified donor T-cells are sometimes used to transiently increase the transplant recipient's absolute number of immune cells; however, these methods rely on the clonal expansion and activation of donor cells and do not restore the diversity of the T- and B-cell repertoire.

In contrast to endogenous bone marrow, the present inventions generally relate to compositions and methods that involve the development of an ectopic bone marrow niche or nodule which forms following the administration or implantation of the compositions disclosed herein to a subject (e.g., a mammalian subject in need thereof). For example, in certain aspects, the present inventions are directed to compositions comprising a porous implantable scaffold material and one or more differentiation factors that specify lymphopoiesis and promote the differentiation of the transplanted HSCs to lymphoid lineage cells (CLP), thereby aiding the reconstitution of the immune system of the subject. The endogenous generation of naïve immune cells capable of mounting immune responses against appropriate antigens thus confers broad, long-term immunity.

The compositions disclosed herein comprise one or more scaffold materials (e.g., a porous implantable scaffold material), which may be administered to or otherwise implanted in a subject. For example, in certain embodiments the compositions disclosed herein comprise one or more growth factors that may be loaded into or encapsulated by a scaffold material and, following its administration to a subject (e.g., an immunocompromised subject), such composition promotes the formation of tissue (e.g., bone tissue) on or around the administered scaffold material to form a nodule that functions as an ectopic bone marrow niche. Accordingly, in certain aspects the scaffold materials disclosed herein provide a delivery vehicle for one or more growth factors, differentiation factors, homing factors, cytokines, chemokines and any other agents.

In certain embodiments, the scaffold material comprises a polymer (e.g., a three-dimensional polymer system). In certain embodiments, the scaffold material is osteoinductive. In certain aspects, the scaffold material is or comprises alginate (e.g., anionic alginate). In some embodiments, the scaffold material is in the form of a hydrogel.

In some embodiments, the scaffold material is in the form of a cryogel. Cryogels are a class of materials with a highly porous interconnected structure that are produced using a cryotropic gelation (or cryogelation) technique. Cryogelation is a technique in which the polymerization-crosslinking reactions are conducted in quasi-frozen reaction solution. During freezing of the macromoner (e.g., MA-alginate) solution, the macromonomers and initiator system (e.g., APS/TEMED) expelled from the ice concentrate within the channels between the ice crystals, so that the reactions only take place in these unfrozen liquid channels. After polymerization and, after melting of ice, a porous material is produced whose microstructure is a negative replica of the ice formed. Ice crystals act as porogens. Pore size is tuned by altering the temperature of the cryogelation process. For example, the cryogelation process is typically carried out by quickly freezing the solution at −20° C. Lowering the temperature to, e.g., −80° C., would result in more ice crystals and lead to smaller pores. In some embodiments, the cryogel is produced by cryo-polymerization of at least methacrylated (MA)-alginate and MA-PEG. In some embodiments, the cryogel is produced by cryo-polymerization of at least MA-alginate, a differentiation factor, and MA-PEG. In some embodiments, the differentiation factor further comprises a tether (e.g., PEG, PEG₂ₖ) and a MA group. In some embodiments, the differentiation factor is DLL4-PEG₂ₖ-MA. In some embodiments, the cryo-polymerization is carried out in the presence of an agent to be encapsulated in the cryogel. In some embodiments, the agent is one or more growth factors, differentiation factors, homing factors, cytokines, and chemokines. In some embodiments, the agent is BMP (e.g., BMP-2).

The cryogel may comprise at least 75% pores, e.g., 80%, 85%, 90%, 91%, 92%, 93%, 94%, 95%, 96%, 97%, 98%, or 99% or more pores. The pores are interconnected. Interconnectivity of the pores permits passage of water (and other compositions such as cells and compounds) in and out of the structure. In a fully hydrated state, the composition comprises at least 90% water (e.g., between 90-99%, at least 92%, 95%, 97%, 99/6, or more) water. For example, at least 90% (e.g., at least 92%, 95%, 97%, 99%, or more) of the volume of the cryogel is made of liquid (e.g., water) contained in the pores. In a compressed or dehydrated hydrogel, up to 50%, 60%, 70% of that water is absent, e.g., the cryogel comprises less than 25% (20%, 15%, 10%, 5%, or less) water.

The cryogels of the invention may comprise pores large enough for a cell to travel through. For example, the cryogel contains pores of 20-500 μm in diameter, e.g., 20-300 μm, 30-150 μm, 50-500 μm, 50-450 μm, 100-400 μm, 200-500 μm. In some cases, the hydrated pore size is 1-500 μm (e.g., 10-400 μm, 20-300 μm, 50-250 μm).

In some embodiments, cryogels are further functionalized by addition of a functional group chosen from the group consisting of: amino, vinyl, aldehyde, thiol, silane, carboxyl, azide, alkyne. Alternatively, the cryogel is further functionalized by the addition of a further cross-linker agent (e.g. multiple arms polymers, salts, aldehydes, etc). The solvent can be aqueous, and in particular acidic or alkaline. The aqueous solvent can comprise a water-miscible solvent (e.g. methanol, ethanol, DMF, DMSO, acetone, dioxane, etc). In some embodiments, one or more functional groups are added to a constitutent of the cryogel (e.g., alginate, PEG) prior to cryogelation. The cryo-crosslinking make take place in a mold and the injectable cryogels can be degradable. The pore size can be controlled by the selection of the main solvent used, the incorporation of a porogen, the freezing temperature and rate applied, the cross-linking conditions (e.g. polymer concentration), and also the type and molecule weight of the polymer used.

In some embodiments, the scaffold material and compositions disclosed herein have not been seeded or otherwise loaded with cells prior to their administration or implantation in a subject. Alternatively, in other embodiments, the scaffold material and compositions disclosed herein have been seeded or otherwise loaded with cells (e.g., HSCs) prior to their administration or implantation in a subject. In yet other embodiments, the scaffold material is selected from the group consisting of polylactic acid, polyglycolic acid, PLGA polymers, alginates and alginate derivatives, polycaprolactone, calcium phosphate-based materials, gelatin, collagen, fibrin, hyaluronic acid, laminin rich gels, agarose, natural and synthetic polysaccharides, polyamino acids, polypeptides, polyesters, polyanhydrides, polyphosphazines, poly(vinyl alcohols), poly(alkylene oxides), poly(allylamines)(PAM), poly(acrylates), modified styrene polymers, pluronic polyols, polyoxamers, poly(uronic acids), poly(vinylpyrrolidone) and any combinations or copolymers thereof. Other exemplary scaffold materials, compositions and methods of their use and preparation are described in U.S. Patent Publication Nos. 2008/0044900, 2013/0331343, 2015/0366956, 2014/0112990, 2014/0227327 and 2015/0359928, which are incorporated by reference herein in their entirety.

The scaffold materials disclosed herein may be further modified, for example, to include compounds or excipients to render such scaffolds materials osteoinductive (e.g., calcium phosphate). Similarly, compounds or excipients may be included in the scaffold material to influence its mechanical properties. For example, to tune the mechanical properties of the scaffold material, polymers such as rigid polycaprolactone (PCL) and soft polyethylene glycol (PEG) can be used in combination with alginate.

The scaffold materials may be used to control the in vivo presentation or release of one or more growth factors, differentiation factors and/or homing factors, for example, upon administration or implantation of the scaffold material or composition. For example, using the carboxylic acid group on the alginate backbone EDC/NHS chemistry may be used to conjugate DLL-4 to the scaffold material. Such presentation or release of one or more growth factors, differentiation factors and/or homing factors may be accomplished by encapsulating or coupling (e.g., covalently binding or coupling) these molecules in or on the scaffold material (e.g., coupling the molecule to the alginate backbone). The spatial and temporal presentation of such molecules may be precisely controlled by fine-tuning the chemical reactions used to couple these molecules, as well as by selecting or altering the physical and chemical properties of the scaffold material. As a result, such scaffold materials are especially useful for controlling the in vivo delivery and/or presentation of one or more molecules (e.g., a growth factor) that may be encapsulated therein or coupled thereto. Accordingly, based upon the selection of one or more growth factors, differentiation factors and/or homing factors, the release of such molecules from the scaffold material may be optimized to achieve and accurately control the behavior (e.g., the proliferation, migration and/or differentiation) of transplanted stem and progenitor cells in vivo.

In certain aspects, following administration or implantation of the compositions disclosed herein, the one or more growth factors contained in such compositions promote the formation of tissue (e.g., bony tissue) on or around the administered composition, thereby forming a nodule that functions as an ectopic bone marrow niche. As used herein, the term “growth factor” generally refers to any biologically active agent, polypeptide, hormone, cytokine, chemokine, or compound which causes or otherwise promotes cellular proliferation, and in certain aspects includes osteogenic growth factors and their analogs. Exemplary growth factors include bone morphogenetic proteins, such as BMP-2, BMP-4, BMP-6, BMP-7, BMP-12 and BMP-14. The one or more of the growth factors may be encapsulated in the scaffold material and released from the scaffold material over an extended period of time (e.g., over about 7-14 days). The one or more of the growth factors may be encapsulated in the scaffold material and released from the scaffold material over about 1-50, 5-25, 10-20, or 17-18 days. In some embodiments, such one or more growth factors (e.g., BMP-2) are released over a sufficient period of time to allow formation of tissue (e.g., a bony tissue) on or around the implanted composition to form a nodule.

The compositions disclosed herein further comprise one or more differentiation factors. As used herein, the term “differentiation factor” broadly refers to any molecule that promotes cellular differentiation. For example, any differentiation factor that promotes the differentiation of stem cells or progenitor cells to one or more lymphoid lineage cells. In certain aspect the differentiation factor promotes the migration of a transplanted cell (e.g., a HSC) to one or more secondary sites, such as the thymus and/or lymph nodes, where such cells then mature. For example, transplanted stem cells may home to or otherwise infiltrate the nodule, where such cells contact a differentiation factor (e.g., a Notch ligand such as DLL-2 and/or DLL-4), and then further migrate to the thymus where they will mature into T-cell. In certain aspect, the differentiation factor promotes the differentiation and maturation of the transplanted cell within the nodule. For example, transplanted stem cells may home to or otherwise infiltrate the nodule, where such cells contact a differentiation factor (e.g., IL-7 or IL-15) and then differentiate within the nodule into a mature B-cell.

In certain aspects, the differentiation factors disclosed herein may be modified as necessary to, for example, optimize the performance of the compositions disclosed herein. In some embodiments, the Notch binding peptide fragment (˜2 kDa) of DLL-1 or DLL-4 may be used instead of the full protein (˜70 kDa) to allow for greater functionalization of the alginate scaffold material. In some embodiments, to further enhance the effect of the scaffold material, angiogenic vascular endothelial growth factor (VEGF) and/or chemotactic SDF-1 can be incorporated.

In some embodiments, the compositions disclosed herein further comprise one or more “homing factors,” which term generally refers to any compositions that promote the migration or homing of transplanted stem cells to the nodule that is formed upon administration or implantation of the compositions disclosed herein. Stem cell transplant is largely possible because hematopoietic stem cells have an innate ability to “home” to the bone marrow from the bloodstream after being infused into a subject. By incorporating, encapsulating or tethering one or more homing factors into the scaffold materials that comprise the compositions disclosed herein, the homing of transplanted stem cells (e.g., HSCs) to the nodule is enhanced. For example, the methods and compositions disclosed herein are useful for enhancing (e.g., increasing) the ability of infused HSCs to home to the ectopic bone marrow niche formed by the nodule from the tissues where such HSCs were infused during transplant (e.g., blood). As used herein, the terms “home” and “homing” mean that the transplanted stem cells (e.g., HSCs or progenitor cells) migrate, move or otherwise concentrate from a first particular tissue or region where they were infused (e.g., the blood), to a second tissue or region where they are needed (e.g., the nodule formed following the implantation of the composition in a subject). Various factors within the endogenous stem cell niche regulate the homing of HSCs to the bone marrow stem cell niche and one or more of such factors may be incorporated into the compositions disclosed herein. One such factor that promotes HSC retention and homing within the endogenous bone marrow is stem cell differentiation factor (SDF-1). Accordingly, in certain aspects, the compositions disclosed herein comprise SDF-1, which promotes homing of transplanted stem cells to the nodule and the subsequent engraftment of such cells in the ectopic bone marrow niche formed by such nodule.

The methods and compositions disclosed herein are useful in connection with stem cell transplantation (e.g., HSC transplant). As used herein, the term “hematopoietic stem cells” or “HSCs” refers to stem cells that can differentiate into the hematopoietic lineage and give rise to all blood cell types such as white blood cells and red blood cells, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells). Stem cells are defined by their ability to form multiple cell types (multipotency) and their ability to self-renew. Hematopoietic stem cells can be identified, for example by cell surface markers such as CD34−, CD133+, CD48−, CD150+, CD244−, cKit+, Sca1+, and lack of lineage markers (negative for B220, CD3, CD4, CD8, Mac1, Gr1, and Terl19, among others).

As used herein, the term “progenitor cells” encompasses pluripotent cells which are committed to the hematopoietic cell lineage, generally do not self-renew, and are capable of differentiating into several cell types of the hematopoietic system, such as granulocytes, monocytes, erythrocytes, megakaryocytes, B-cells and T-cells, including, but not limited to, short term hematopoietic stem cells (ST-HSCs), multi-potent progenitor cells (MPPs), common myeloid progenitor cells (CMPs), granulocyte-monocyte progenitor cells (GMPs), megakaryocyte-erythrocyte progenitor cells (MEPs), and committed lymphoid progenitor cells (CLPs). The presence of hematopoietic progenitor cells can be determined functionally as colony forming unit cells (CFU-Cs) in complete methylcellulose assays, or phenotypically through the detection of cell surface markers (e.g., CD45−, CD34+, Teri 19−, CD16/32, CD127, cKit, Sca1) using assays known to those of skill in the art.

Certain aspects of the methods disclosed herein comprise administering or otherwise transplanting stem cells to a subject in need, such that the administered stem cells engraft in the ectopic bone marrow niche of the nodules (e.g., the bony nodules) that form following administration of the compositions disclosed herein to a recipient subject. As used herein “engrafting” and “engraftment” of a stem cell, including hematopoietic stem cells, means placing the stem cell into an animal, e.g., by injection, wherein the stem cell persists in vivo. This can be readily measured by the ability of the stem cell, for example, to contribute to the ongoing immune cell and/or blood cell formation. Successful stem cell transplantation depends on the ability to engraft sufficient quantities of transplanted stem cells in the tissues of the subject and the compositions and method disclosed herein therefore increase the number or volume of sites and tissues where such transplanted stem cells may engraft.

As used herein, the term “administering,” generally refers to the placement of the compositions described herein into a subject (e.g., the parenteral placement or implantation of such compositions into a subject) by a method or route which results in migration or homing of transplanted stem cells to the ectopic bone marrow niche of the nodules. In certain aspects, the compositions disclosed herein are administered to or implanted in multiple sites of the subject, for example, prior to undergoing a stem cell transplant. In certain embodiments, the compositions disclosed herein are administered at a site near the lymphatic system of the subject (e.g., in proximity to one or more of the neck, groin, and underarms of the subject). In certain aspects, following reconstitution of the subject's immune system, the nodules disclosed herein are removed (e.g., by surgical excision).

In certain embodiments, the compositions and methods disclosed herein are useful for the treatment of any disorder, disease, condition, or complication in which transplantation of hematopoietic stem cells and/or progenitor cells is desirable. Examples of such disorders include hematological malignancies and non-malignant hematological diseases. The compositions and methods disclosed herein are also useful for reconstituting T-cells and B-cells and accordingly can be broadly applied to other diseases in which immunodeficiency is implicated, such as age-related vaccine failure, autoimmune disorders (e.g., rheumatoid arthritis and diabetes), infectious disease, and others.

As used herein, the term “subject” means any human or animal. In certain aspects, the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing (e.g., all of the above), but excluding one or more groups or species such as humans, primates or rodents. In certain embodiments, the subject is a mammal (e.g., a primate or human). In some embodiments, the mammal is a human, a non-human primate, a mouse, a rat, a dog, a cat, a horse, or a cow, and is not limited to these examples. Mammals other than humans can be advantageously used, for example, as subjects that represent animal models of, for example, a hematological malignancy. In addition, the methods described herein can be used to treat domesticated animals and/or pets. A subject can be male or female.

In certain embodiments, a subject can be one who has been previously diagnosed with or otherwise identified as suffering from or having a condition, disease, or stem cell disorder. A “subject in need” of treatment for a particular condition (e.g., a stem cell disorder) can be a subject having that condition, diagnosed as having that condition, or at increased risk of developing that condition relative to a given reference population. In some embodiments, the methods of treatment described herein comprise selecting a subject diagnosed with, suspected of having, or at risk of developing a hematological malignancy or being immunocompromised. In some embodiments, the methods described herein comprise selecting a subject diagnosed with, suspected of having, or at risk of developing a non-malignant disease, for example a non-malignant disease described herein.

In some embodiments, the methods disclosed herein accelerate the reconstitution of the subject's immune system from endogenous progenitor HSCs. In some embodiments, the methods disclose herein accelerate reconstitution from endogenous progenitor HSCs by about 1.5, 1.6, 1.7, 1.8. 1.9. 2, 2.1, 2.2, 2.3, 2.4, 2.5, 3, or 4 orders of magnitude or more. In some embodiments, the methods disclose herein accelerate reconstitution from endogenous progenitor HSCs by over 2 orders of magnitude.

In some embodiments, the methods disclosed herein accelerate the reconstitution of the subject's immune system from transplanted progenitor HSCs. In some embodiments, the methods disclose herein accelerate reconstitution from transplanted progenitor HSCs by about 1.5, 1.6, 1.7, 1.8, 1.9.2, 2.1, 2.2, 2.3, 2.4, 2.5, 3, or 4 orders of magnitude or more. In some embodiments, the methods disclose herein accelerate reconstitution from transplanted progenitor HSCs by over 2 orders of magnitude.

It is to be understood that the invention is not limited in its application to the details set forth in the description or as exemplified. The invention encompasses other embodiments and is capable of being practiced or carried out in various ways. Also, it is to be understood that the phraseology and terminology employed herein is for the purpose of description and should not be regarded as limiting.

While certain agents, compounds, compositions and methods of the present invention have been described with specificity in accordance with certain embodiments, the following examples serve only to illustrate the methods and compositions of the invention and are not intended to limit the same.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications and other reference materials referenced herein to describe the background of the invention and to provide additional detail regarding its practice are hereby incorporated by reference.

### Examples

### Example 1—Synthetic Materials to Induce the Formation of Hone Nodule

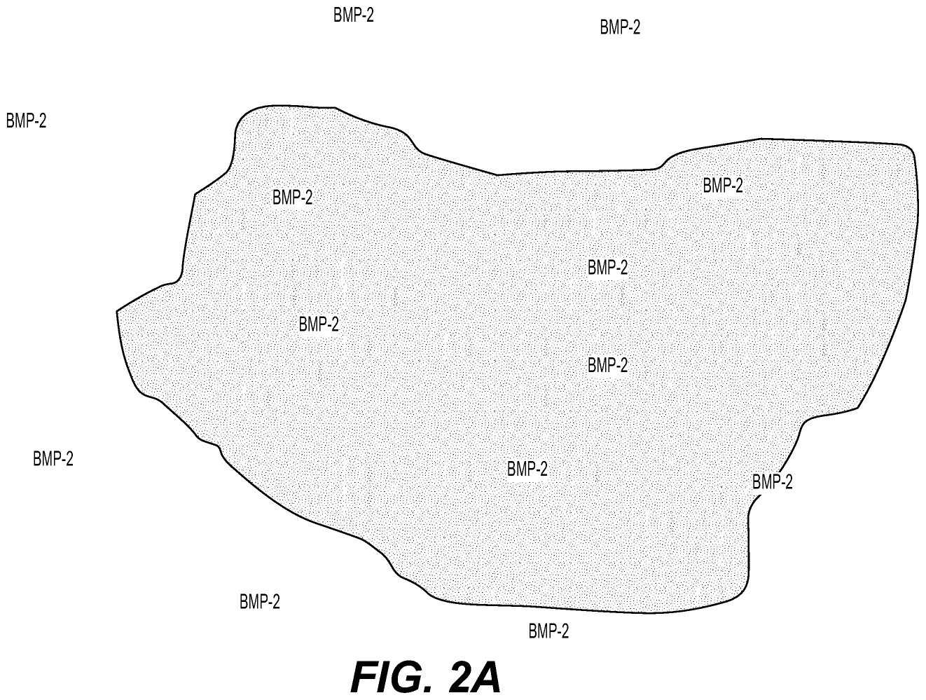

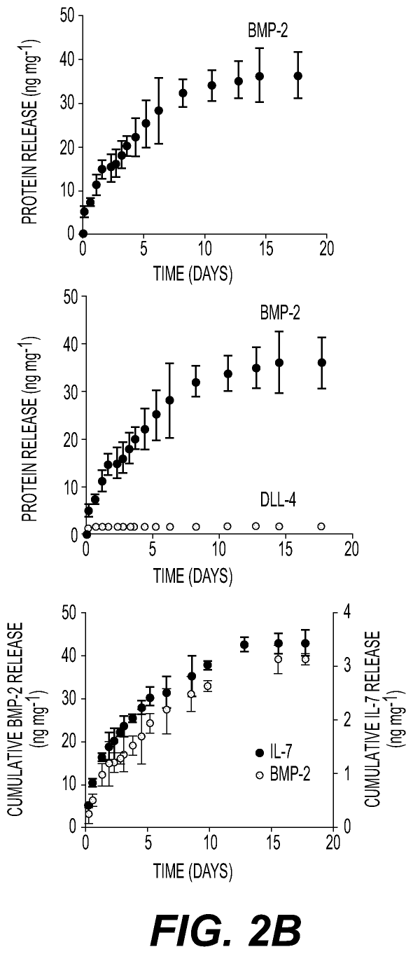

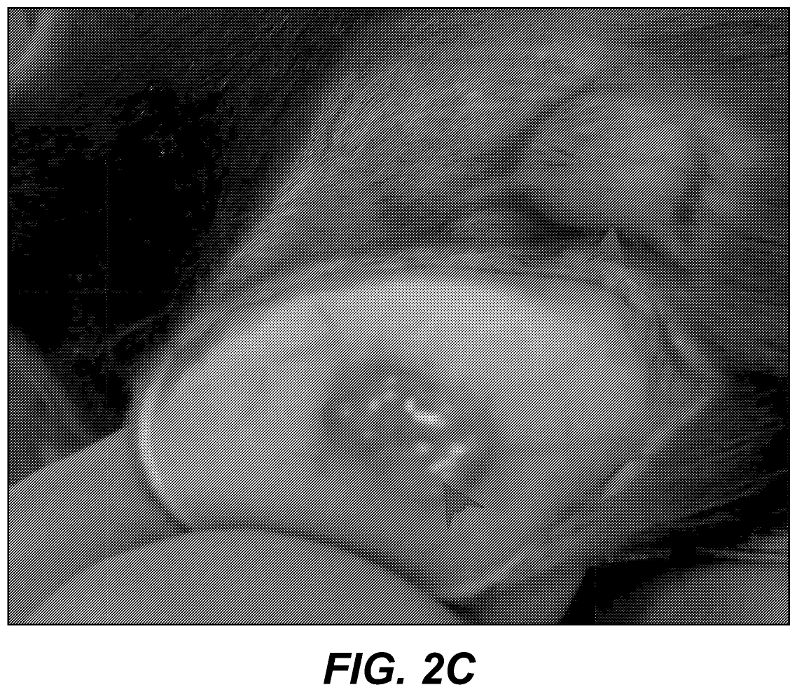

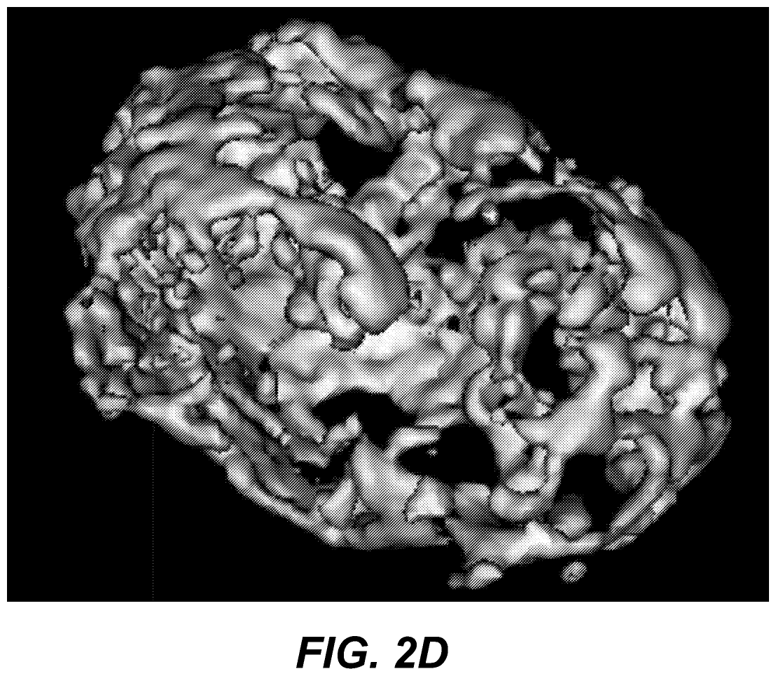

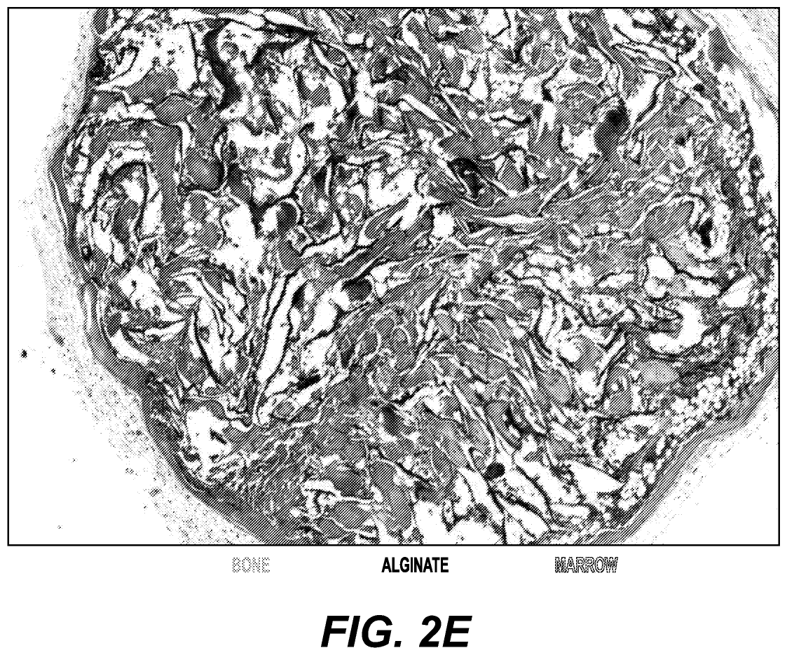

The present inventors used alginate, a natural anionic polysaccharide, as a hydrogel scaffold material and which was adapted to incorporate a cationic, potent bone forming growth factor, bone morphogenetic protein (BMP-2) to create a bone nodule, as illustrated in FIGS. 2A-2E. The loading and release behavior of the BMP-2 growth factor from the alginate was optimized (e.g., total amount at injection and cross-link density of alginate to control release) with the goal of creating a functional, active bone nodule within 1-2 weeks after subcutaneous injection in vivo in a mouse.

Both T- and B-cells arise from a specific type of HSC progenitor cell, the common lymphoid progenitor (CLP). Within the bone marrow, the Notch pathway is critical for lymphocyte specification. CLPs exposed to the Notch ligand delta-like ligand 1 or 4 (DLL-1/4) traffic to the thymus and differentiate into T-cells. CLPs in the bone marrow exposed to interleukin-7 (IL-7) differentiate into B-cells. To specify lymphocyte differentiation of the transplanted HSCs that homed to the ectopic bone marrow, the present inventors assessed the effect of incorporating the Notch ligand DLL-4 within the ectopic bone marrow. Using the carboxylic acid group on the alginate backbone, EDC/NHS chemistry was used to conjugate DLL-4 in an effort to direct T-cell differentiation of the HSCs.

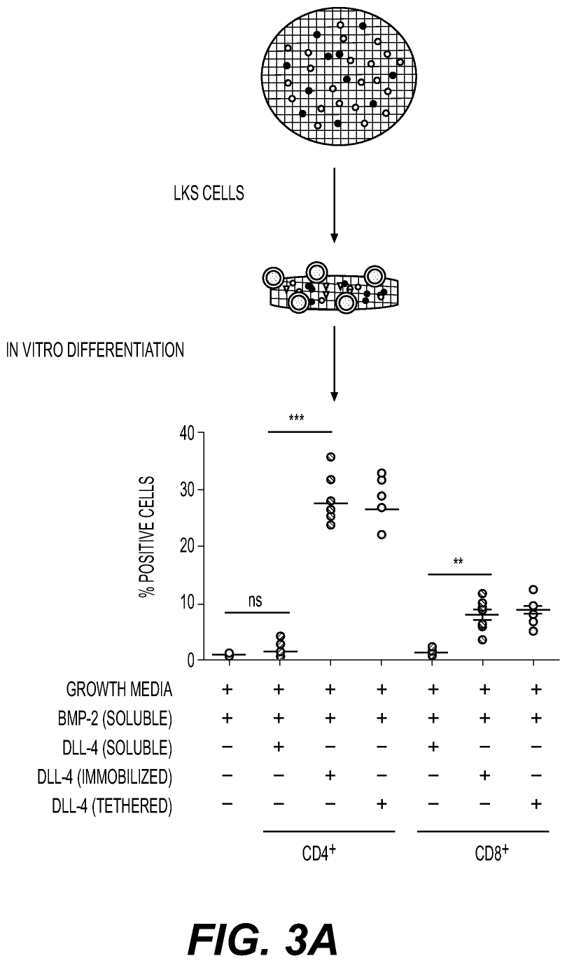

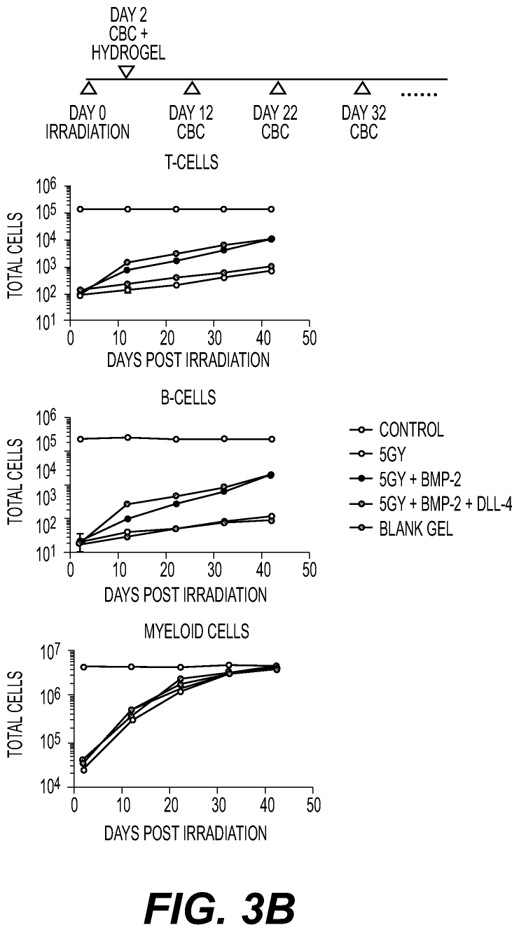

The in vitro differentiation potential of hydrogels containing the growth factor BMP-2 and the differentiation factor DLL-4 was tested using Lin-c-kit+Sca-1+ (LKS) cells, the putative hematopoietic stem and progenitor cells. The present inventors assayed for differentiation into CD4+ and CD8+ T-cells and compared different strategies to incorporate the DLL-4 (FIG. 3A). It was observed that the conjugation and tethering of DLL-4 was necessary for biological activity. The present inventors then tested a subset of the best performing gels in sub-lethally irradiated mice and observed an acceleration in the rate of T- and B-cell production, as compared to the untreated group, as illustrated in FIG. 3B.

In addition to the foregoing studies, the present inventors will homogenize and section the resulting ectopic bone marrow nodule to determine cell populations. The level of stromal cell derived factor-1 (SDF-1) for HSC chemotaxis will also be measured, and histological assessment of the vascularity and cellularity of the ectopic bone marrow nodule will be compared with healthy marrow. The present inventors will further optimize the system by conjugating DLL-4 to the alginate backbone over a range of molar ratios (e.g., 10⁻⁹ to 10⁻⁶). Furthermore, to exclusively drive B-cell development in the bone marrow, the present inventors will incorporate soluble IL-7 in the hydrogel over a range of doses (100-500 ng) instead of DLL-4, with the goal of sustained release of the soluble IL-7 for at least 1 week. Flow cytometry will be used to follow lymphoid progenitors as they differentiate, mature and migrate through the bone marrow, peripheral lymphoid organs, blood, and spleen.

### Example 2—Characterizing Lymphocyte Reconstitution and the Immune Repertoire after HSTC

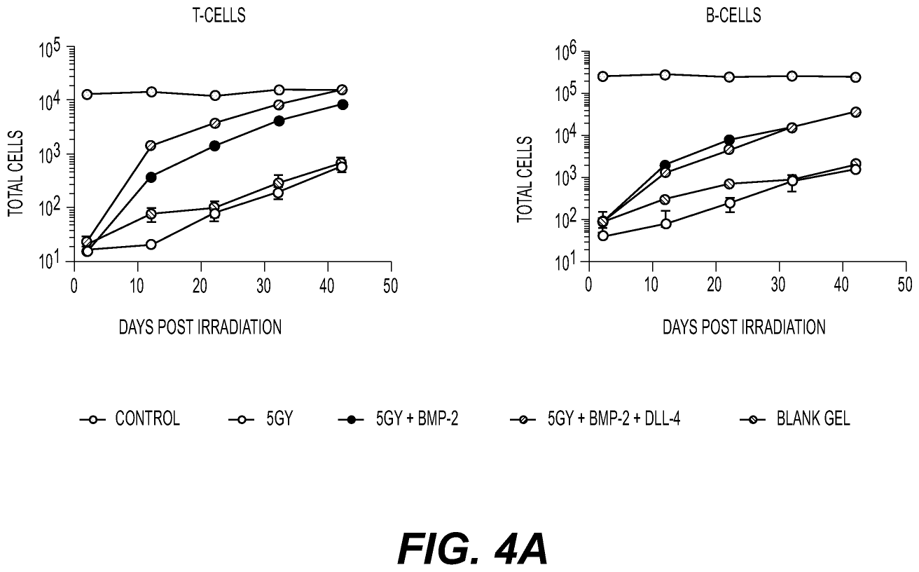

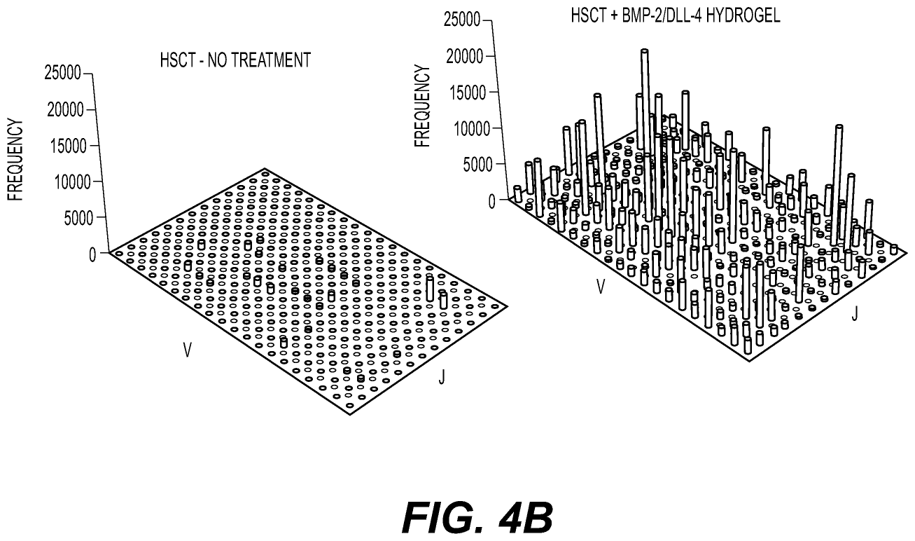

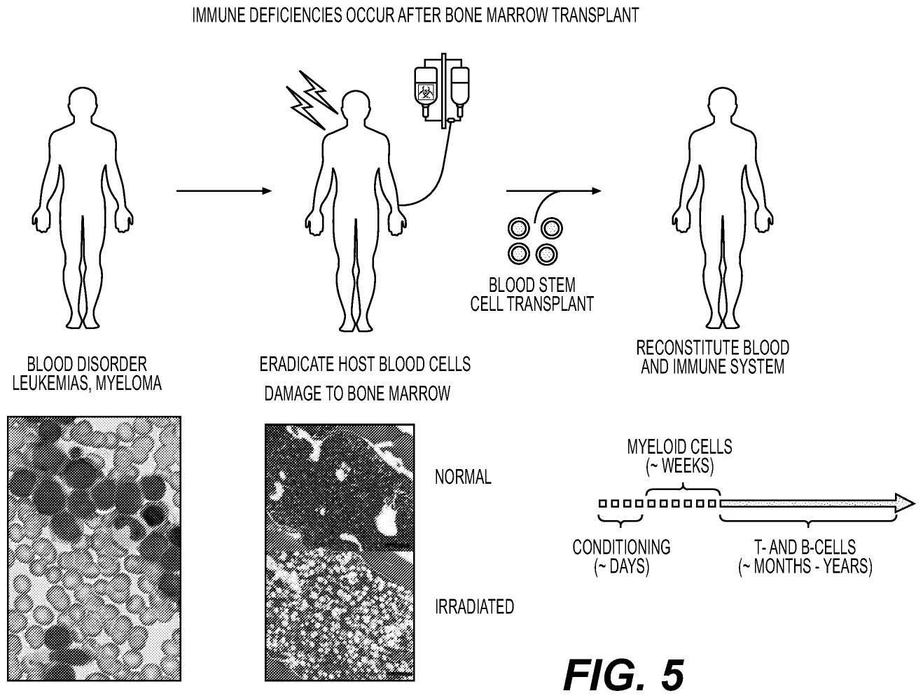

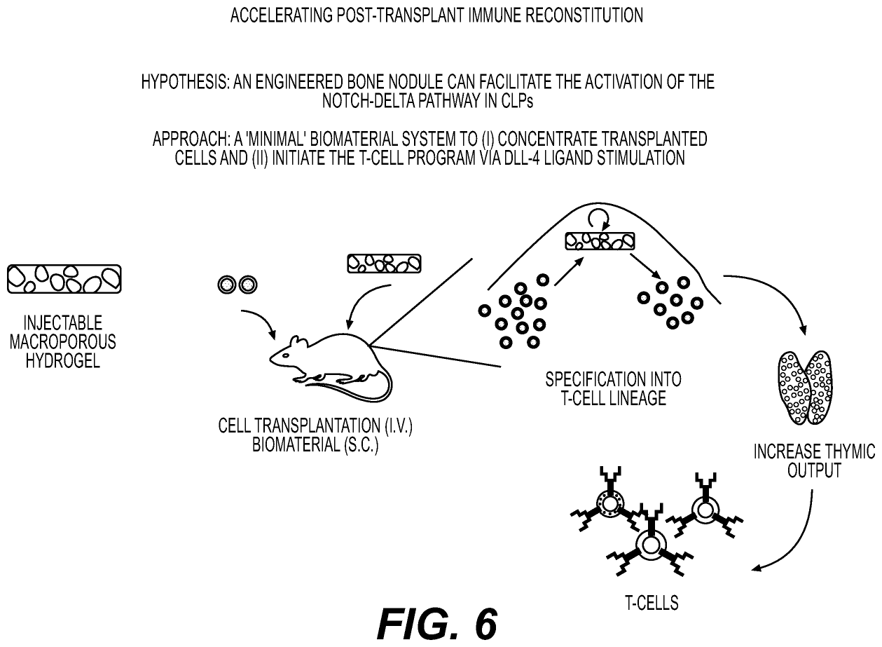

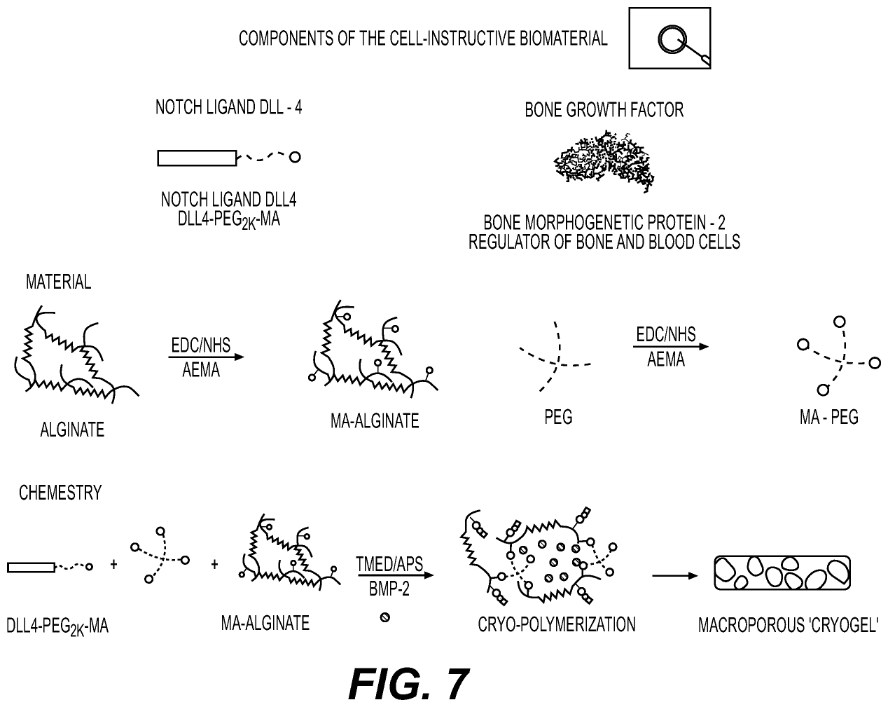

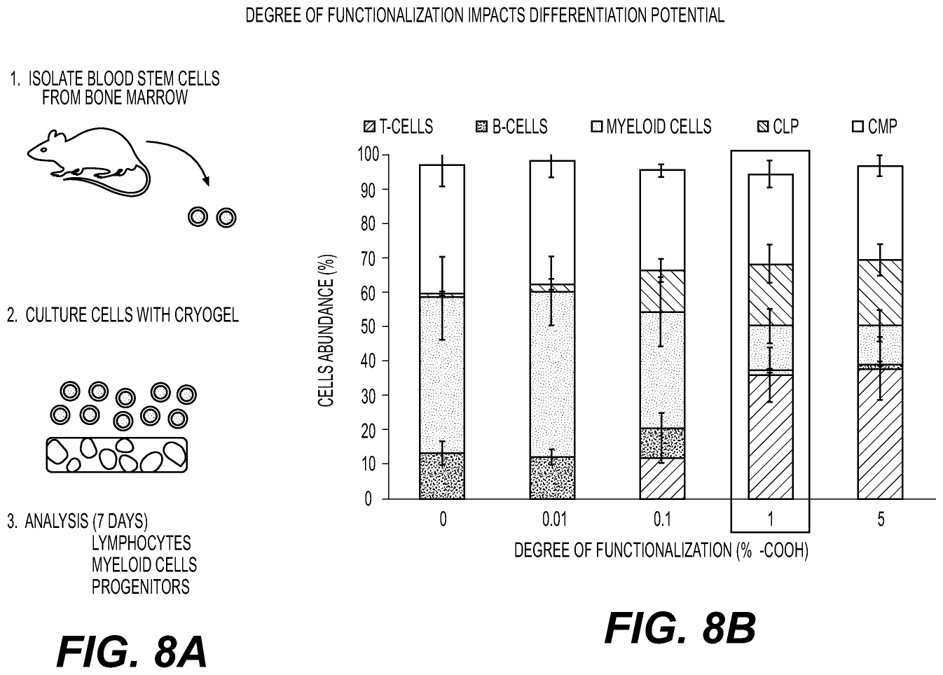

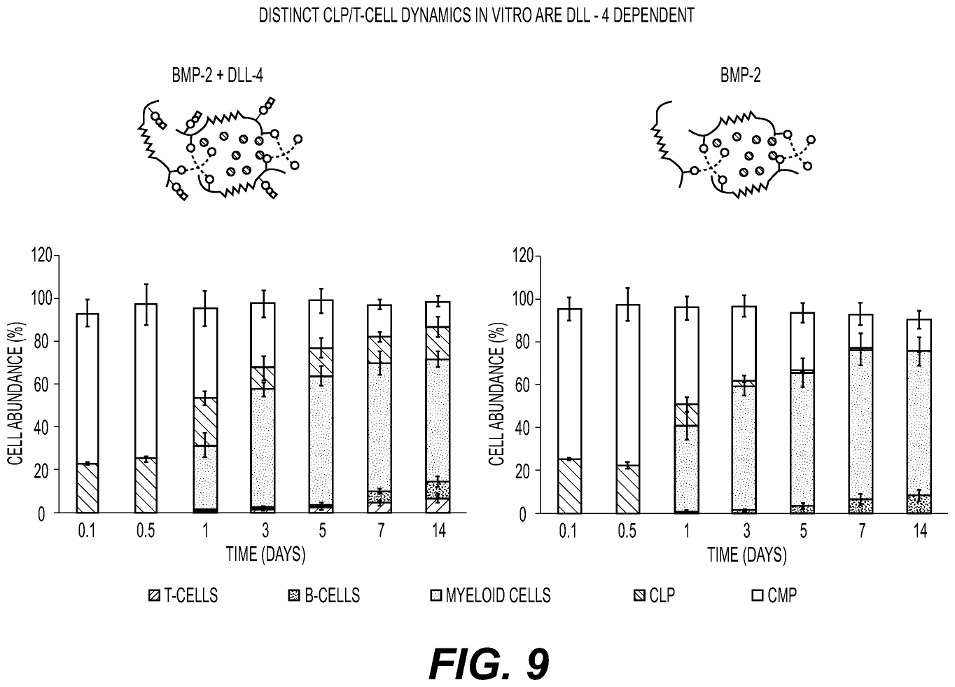

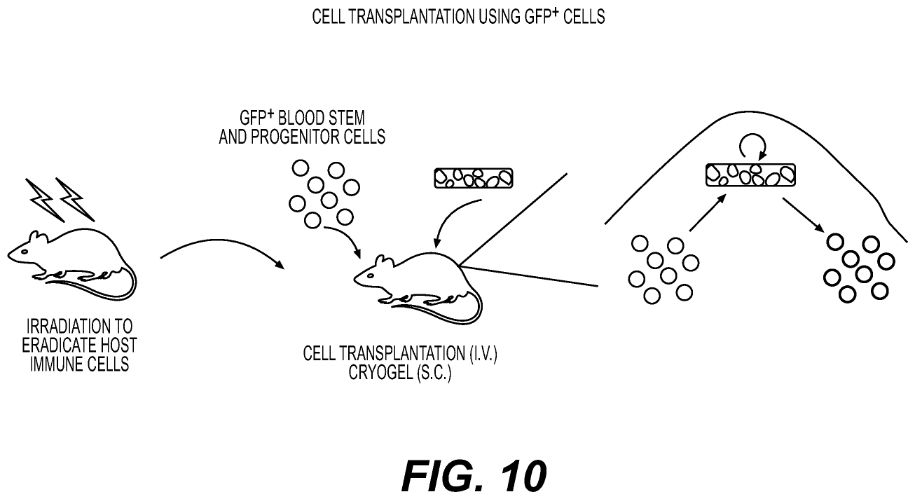

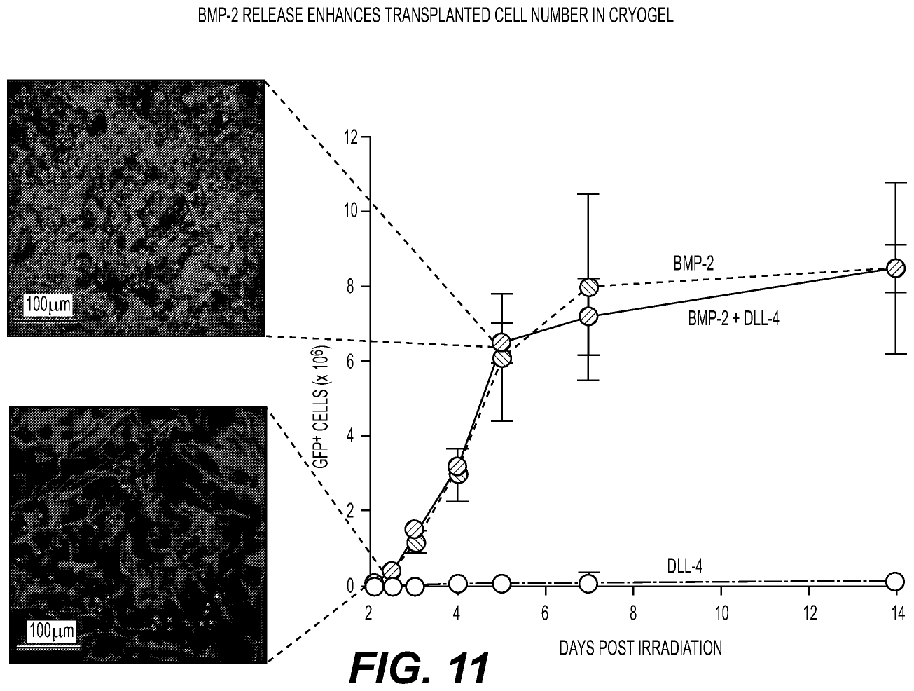

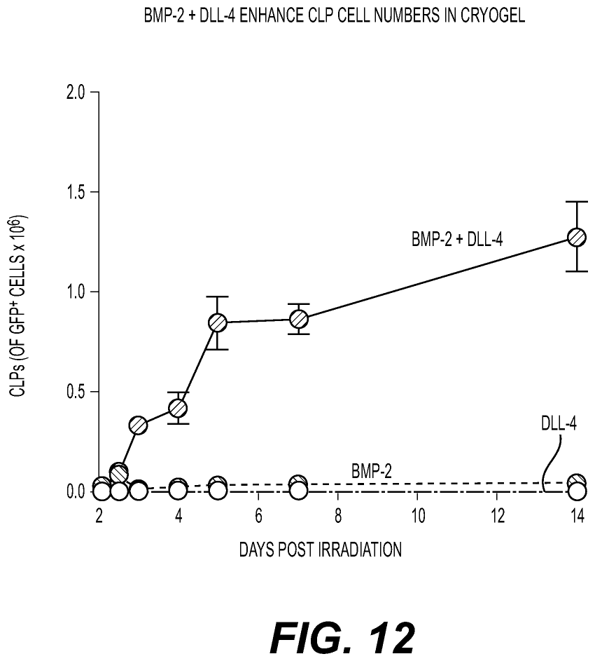

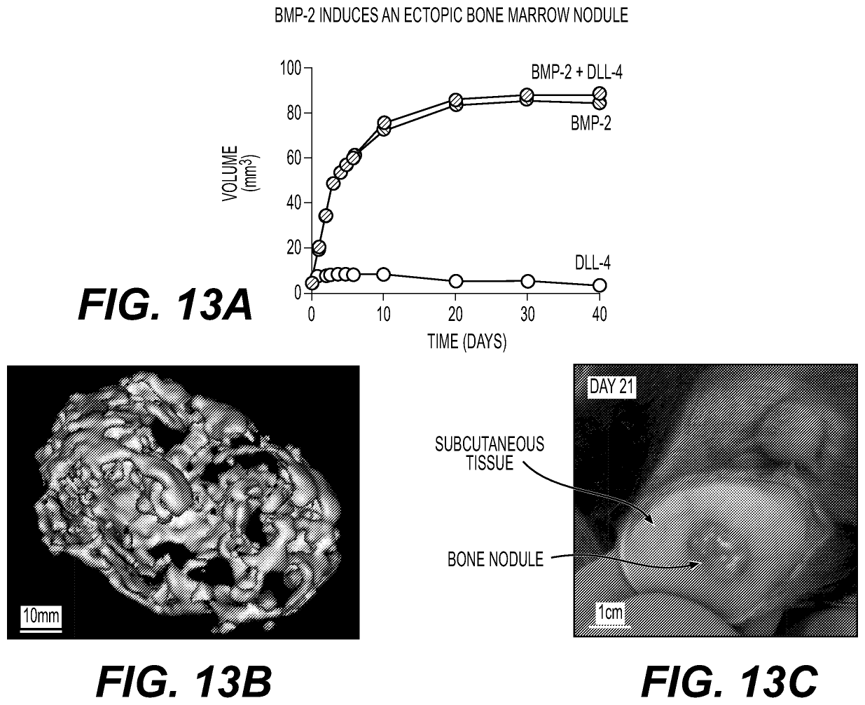

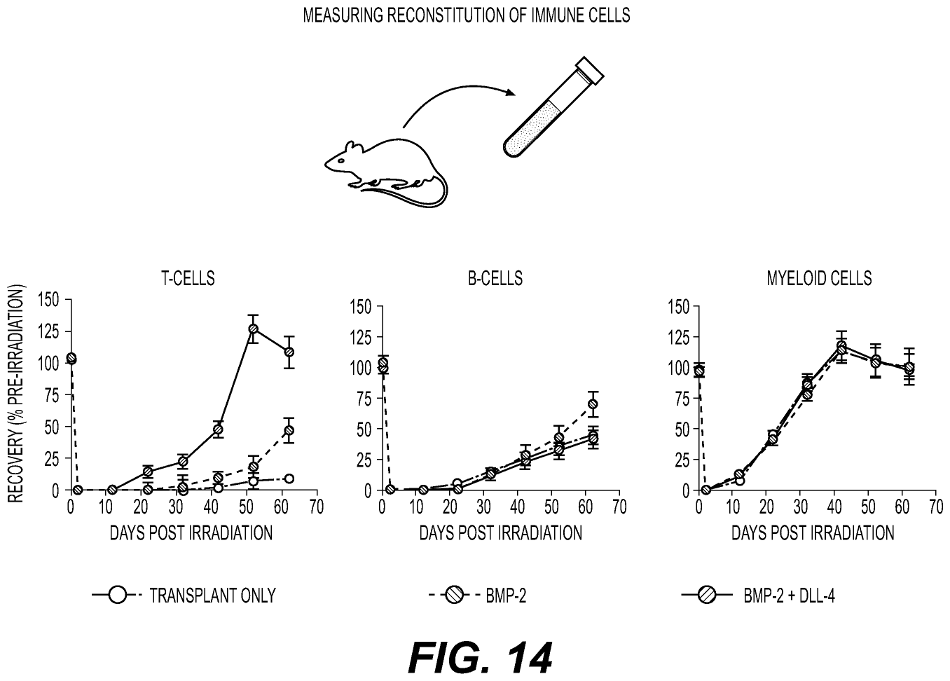

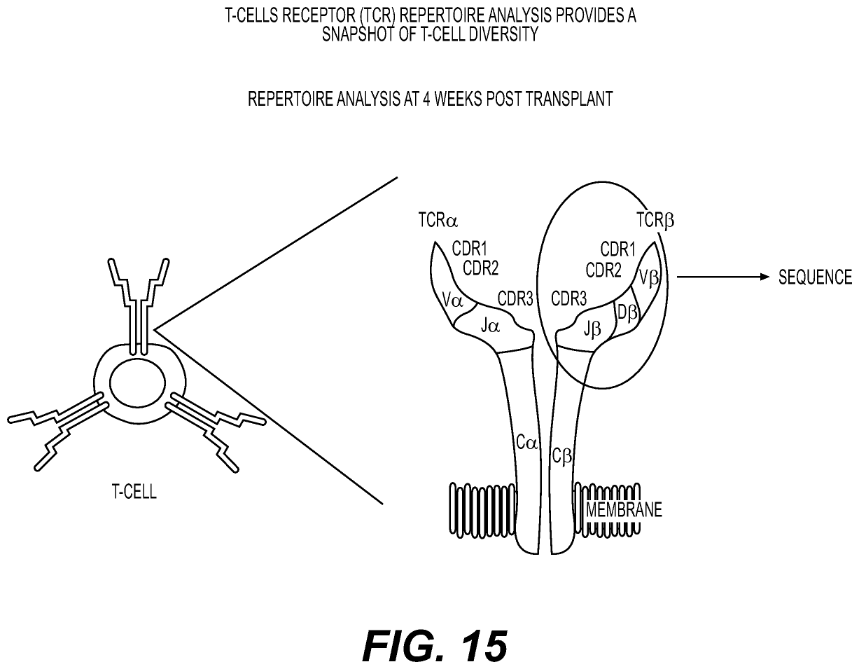

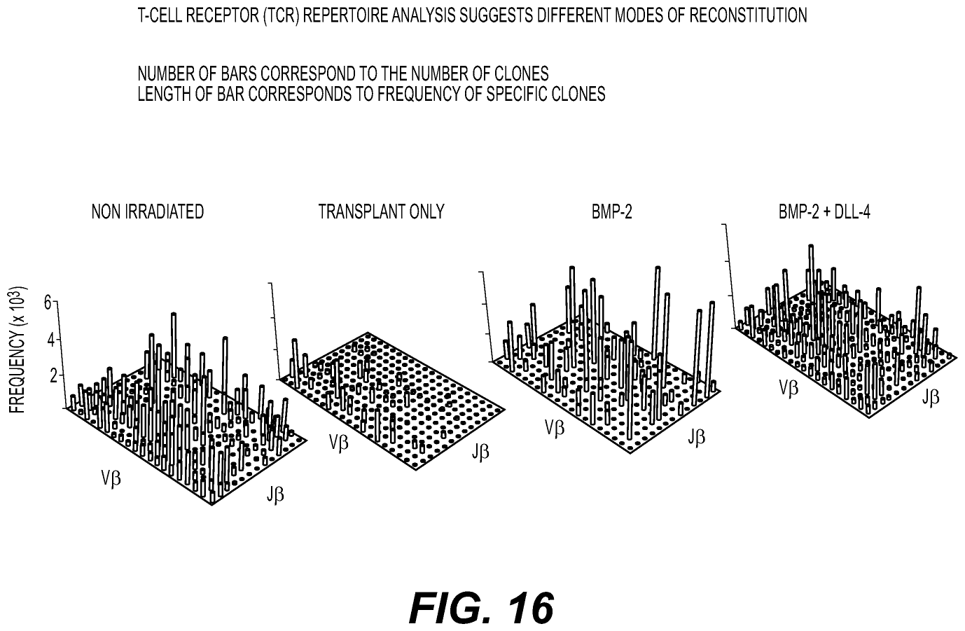

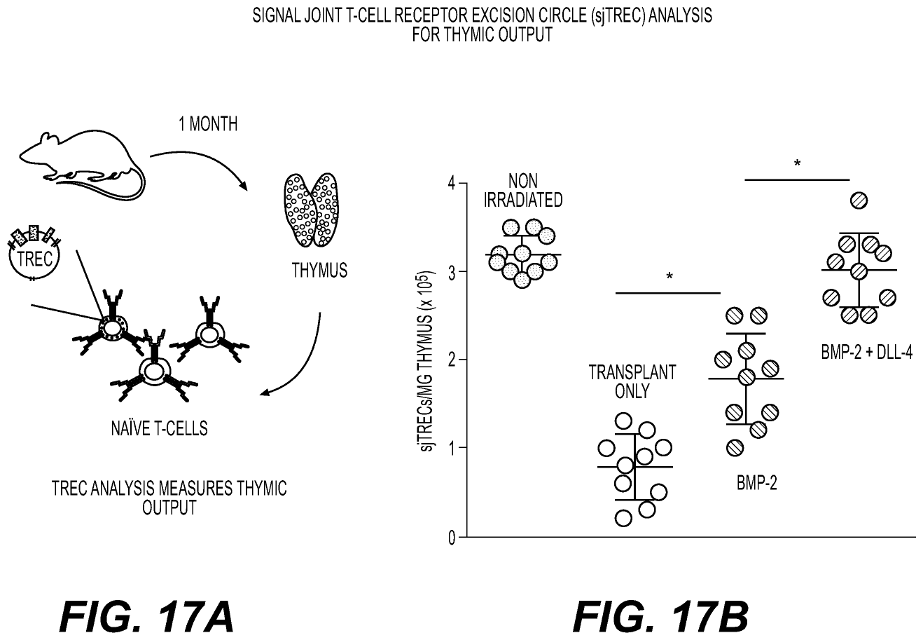

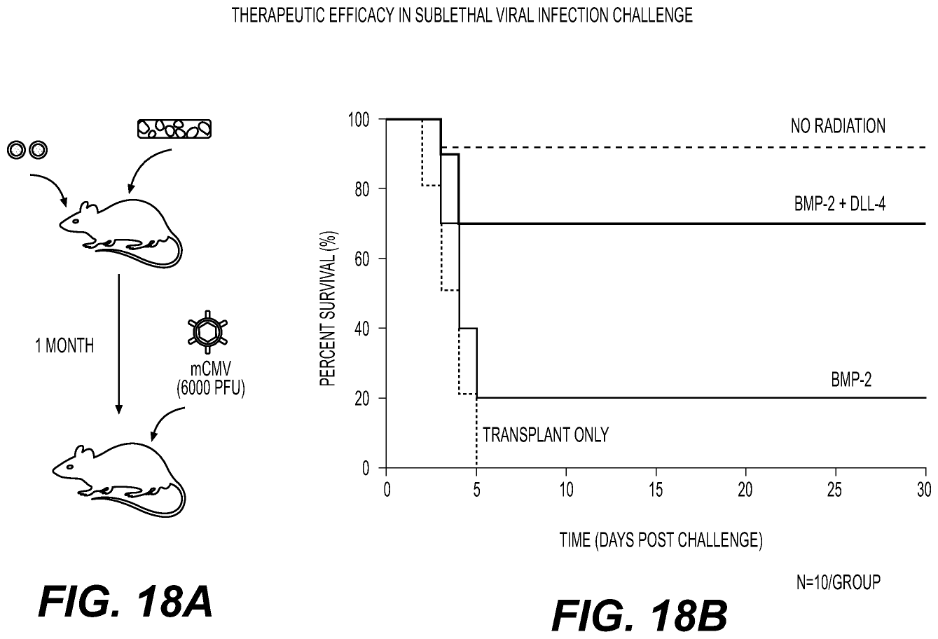

Naïve T-cells and B-cells are essential for continuous responses to unfamiliar pathogens via cell-mediated and humoral immunity respectively. The reconstitution of naïve lymphocytes derived from donor cells after HSCT were examined. Bone marrow was harvested from donor mice and T- and B-cell depleted (>95%) bone marrow was transplanted into lethally irradiated congenic recipient mice and followed reconstitution of the T- and B-cells, as illustrated in FIG. 4A. By sequencing the T-cell receptor (TCR) of CD3+ cells and examining the frequency and distribution of the variable (V) and joining (J) segments of the TCR gene, the diversity of the TCR was determined, as illustrated in FIG. 4B.

The present inventors will mimic transplant manipulations that are performed in the clinic, and examine using whole bone marrow or purified LKS cells from bone marrow. Along with the transplanted cells, the best performing hydrogel scaffold materials identified from the studies described in Example 1 will be tested. Flow cytometry and complete blood counts will be used to analyze T-cells and B-cells in the hematopoietic compartments. Additionally, the present inventors will use the T-cell receptor excision circle (TREC) assay to measure the output of naïve T-cells from the thymus and assess the naïve CD3+CD4+/CD8+CD62L^{hi} population. Similarly, the present inventors will assess the reconstitution of naïve B-cells (B220+CD40+CD84+) in the bone marrow and the B-cell receptor repertoire.

### Example 3 Macroporous Hydrogel with DLL-4, SDF-1 and BMP-2

UP sodium alginate (ProNova Biomedical) was functionalized with methacrylate groups to prepare methacrylated alginate (MA-Alg). 1-10 nmol of delta-like ligand-4 (DLL-4, R&D systems) was conjugated to MA-Alg using EDC-NHS coupling. Injectable macroporous hydrogels were synthesized by low-temperature redox-induced free-radical polymerization of MA-alginate and 4-arm methacrylated polyethylene glycol (MΛ-PEG) to prepare 2.5 wt % hydrogels. Bone morphogenetic protein-2 (BMP-2, R&D systems) and stem-cell differentiation factor-1 (SDF-1, R&D systems) were added to the mixture prior to cryopolymerization. Differentiation of bone marrow isolated Lin⁻c-kit⁺Sca-1⁺(LKS) cells into CD4⁺ and CD8⁺ T-cells was used to assess the bioactivity of the DLL-4. Sub-lethal and lethal irradiation in C57BL/6 mice was used to mimic immunodeficiency and myeloablative transplant conditioning respectively. Transplants were performed into the CD45.2⁺ mouse strain and monitored for donor-derived cells from congenic CD45.1⁺B6.SJL mice. Hydrogels were injected subcutaneously and the development of a bone nodule and the associated hematopoietic niche environment was monitored using micro-computer tomography (μCT) and histology respectively. Recovery of immune cells in peripheral blood was periodically monitored using FACS analysis. Sequencing of the T-cell receptor (TCR) was used to determine the diversity of the T-cell repertoire.

In the MA-Alg/MA-PEG hydrogel, DLL-4 remained tethered to the scaffold whereas BMP-2 and SDF-1 co-released over a period of 2.5 weeks. In vitro, the hydrogel tethered DLL-4 differentiated Lin⁻c-kit⁺Sca-1⁺(LKS) cells into naïve CD4⁺ and CD8⁺ cells and was comparable to native DLL-4 absorbed on TCPS. In vivo, scaffolds induced the formation of a subcutaneous bony nodule in mice. A histological analysis of the injected hydrogel revealed the formation of a bony nodule with bone marrow, populated with lymphoid progenitors within 2 weeks after injection. In sub-lethally irradiated mice, the BMP-2/DLL-4 hydrogel accelerated the reconstitution of T- and B-lymphocytes by over two orders of magnitude within 3 weeks by recruiting and driving lymphocyte differentiation of the endogenous progenitor HSCs. In lethally irradiated mice that were transplanted, the hydrogel served as a preferential site for transplanted progenitor HSC engraftment and induced their differentiation into T- and B-cells. Recovery of the adaptive immune system followed a similar trend to that of the sub-lethally irradiated mice. The hydrogel-mediated expansion of the T-competent progenitor pool resulted in an increase in the thymic output of transplanted mice and corresponded to an increase in the naïve T-cells pool. Sequencing of the TCR revealed an increase in the frequency and diversity, as measured by the recombination of the variable (V) and joining (J) segments of the TCR gene, of the T-cell repertoire, mediated by the hydrogel.

The results indicate that a programmable biomaterial with biological cucs can recapitulate aspects of the bone marrow stroma. By increasing the available sites of donor cell engraftment and providing the cues for lymphopoiesis, the reconstitution of adaptive immunity can be accelerated after HSCT, which can potentially decrease HSCT-associated immunological complications and improve the treatment of hematological disorders.

1. A composition comprising a porous implantable scaffold material, one or more growth factors and one or more differentiation factors, and/or one or more homing factors.

2. The composition of claim 1, wherein:
(i) the material is a hydrogel;
(ii) the material is a cryogel; and/or
(iii) the material is selected from the group consisting of polylactic acid, polyglycolic acid, PLGA polymers, alginates and alginate derivatives, polycaprolactone, calcium phosphate-based materials, gelatin, collagen, fibrin, hyaluronic acid, laminin rich gels, agarose, natural and synthetic polysaccharides, polyamino acids, polypeptides, polyesters, polyanhydrides, polyphosphazines, poly(vinyl alcohols), poly(alkylene oxides), poly(allylamines)(PAM), poly(acrylates), modified styrene polymers, pluronic polyols, polyoxamers, poly(uronic acids), poly(vinylpyrrolidone) and any combinations or copolymers thereof.

3-5. (canceled)

6. The composition of claim 1, wherein:
(i) one or more of the growth factors comprise a bone morphogenetic protein (BMP);
(ii) one or more of the growth factors are encapsulated in the material; and/or (iii) one or more of the growth factors are released from the material over about 7-30 days.

7. The composition of claim 6, wherein one or more of the growth factors are selected from the group consisting of BMP-2, BMP-4, BMP-6, BMP-12 and BMP-14.

8-10. (canceled)

11. The composition of claim 1, wherein
i) one or more of the differentiation factors bind to a Notch receptor;
ii) one or more of the differentiation factors are covalently bound to the material or covalently bound to a tether that is covalently bound to the material; and/or
iii) one or more of the differentiation factors comprise a cytokine.

12. The composition of claim 11, wherein the Notch receptor is selected from the group consisting of Notch-1, Notch-2, Notch-3 and Notch-4.

13. The composition of claim 11, wherein one or more of the differentiation factors are selected from the group consisting of Delta-like1, Delta-like3, Delta-like4, Jagged1 and Jagged2.

14-15. (canceled)

16. The composition of claim 11, wherein the cytokine comprises interleukin-7 (IL-7).

17. The composition of claim 11, wherein the cytokine is encapsulated in the material; and/or wherein the cytokine is released from the material over about 7-30 days.

18. (canceled)

19. The composition of claim 1, wherein:
(i) one or more of the homing factors comprise stem cell differentiation factor (SDF-1);
(ii) one or more of the homing factors are encapsulated in the material; and/or
(iii) one or more of the homing factors are released from the material over about 7-30 days.

20-21. (canceled)

22. A method of aiding the reconstitution of the immune system ° Ca subject in need thereof, the method comprising administering to the subject the composition of claim 1, wherein:
the one or more growth factors promote formation of tissue on or around the administered scaffold material to form a nodule;
the one or more differentiation factors promote the differentiation of the transplanted stem cells to one or more lymphoid or myeloid lineage cells; and
the one or more homing factors promote the infiltration of transplanted stem cells to the nodule,
thereby aiding the reconstitution of the immune system of the subject.

23. A method of forming an ectopic hematopoietic stem cell niche in a subject in need thereof, the method comprising administering to the subject the composition of claim 1, wherein:
the one or more growth factors promote formation of tissue on or around the administered scaffold material to form a nodule;
the one or more differentiation factors promote the differentiation of the transplanted stem cells to one or more lymphoid or myeloid lineage cells; and
the one or more homing factors promote the infiltration of transplanted hematopoietic stem cells to the nodule,
thereby forming an ectopic hematopoietic stem cell niche in the subject.

24. A method of improving engraftment of a transplanted hematopoietic stem cell in a stem cell niche of a subject in need thereof, the method comprising administering to the subject the composition of claim 1, wherein:
the one or more growth factors promote formation of tissue on or around the administered scaffold material to form a nodule;
the one or more differentiation factors promote the differentiation of the transplanted stem cells to one or more lymphoid or myeloid lineage cells; and
the one or more homing factors promote the infiltration of the transplanted hematopoietic stem cells to the nodule,
thereby improving engraftment of the transplanted hematopoietic stem cell in the stem cell niche of the subject.

25. A method of increasing sites for transplanted stem cells to engraft in a subject in need thereof, the method comprising administering to the subject the composition of claim 1:
the one or more growth factors promote formation of tissue on or around the administered scaffold material to form a nodule;
the one or more differentiation factors promote differentiation of the transplanted stem cells to one or more lymphoid or myeloid lineage cells; and
the one or more homing factors promote the infiltration of the transplanted stem cells to the nodule,
thereby increasing sites for the transplanted stem cells to engraft in the subject.

26-47. (canceled)

48. The method of claim 22, wherein one or more of the differentiation factors promote lymphopoiesis in the transplanted stem cells.

49. The method of claim 22, wherein the subject has undergone a stem cell transplant.

50. The method of claim 22, wherein the subject is immunocompromised.

51. The method of claim 22, wherein one or more of the lymphoid and myeloid lineage cells are CD4+, CD8+, and Mac-1+/GR-1+.
